(19) United States
(12) Patent Application Publication
(10) Pub. No.: US 2017/0320106 A1
(43) Pub. Date: Nov. 9, 2017

(54) ELECTRONIC-DEVICE SANITIZER PRODUCT AND METHOD

(71) Applicants: Casey Hirsch, New Orleans, LA (US); William Noveck, New Orleans, LA (US)

(72) Inventors: Casey Hirsch, New Orleans, LA (US); William Noveck, New Orleans, LA (US)

(21) Appl. No.: 15/148,415

(22) Filed: May 6, 2016

(51) Int. Cl. B08B 1/00; C11D 3/382; C11D 3/22; C11D 3/20; B08B 3/08; C11D 3/48; A61L 2/18

(52) U.S. Cl. CPC B08B 1/006; C11D 3/48; C11D 3/382; A61L 2/18; C11D 3/2065; B08B 3/08; C11D 3/222

(57) ABSTRACT

An electronic-device sanitizer product and method for sanitizing cellphones, smartphones, and other portable electronic devices, that is easy to carry, easy to find when needed, and easy to use, allowing frequent cleaning, having an alcohol-free sanitizing liquid, a misting sprayer, and a microfiber material for distributing and absorbing the sanitizing liquid, all enclosed in a slim tube-shaped package that can be carried on a keyring. Optionally, the sanitizing liquid can be provided with any of a number of distinctive scents that will be imparted to the electronic device.

### BACKGROUND

This invention provides an electronic-device sanitizer product and method for sanitizing cellphones, smartphones, and other portable electronic devices.

Infectious microbial agents on a cellphone or other portable electronic device can cause flu, pinkeye, diarrhea, and other diseases. Average cellphones have been found to have 25,107 bacteria per square inch sitting on their surfaces are considered to have about ten times more disease-causing bacteria than a typical toilet seat. Bacteria strains found on cell phones include E. coli and Staphylococcus aureus, among others, and one in six phones have fecal matter on them. In addition to these infectious agents, cellphones and other portable electronic devices are susceptible to finger, hand, and face prints and smudges.

This causes a problem for users of cellphones and other portable electronic devices, and the number of such users is large and increasing rapidly. Mobile accessory global revenues are forecast to reach $101 billion in 2020. In 2015, smartphone subscriptions increased to $3.4 billion, and are forecast to increase to 6.4 billion by 2021.

The screens of many cellphones and smartphones are covered with semi-permanent films to prevent scratching of the glass. The edges of such protective films against the glass screen provide a haven for detritus and infectious agents to accumulate, and thus are in need of sanitizing. But many of the existing sanitizing products and methods tend to seep under and loosen such protective films, or to discolor or degrade them.

The existing products and methods for sanitizing electronic devices suffer from a number of flaws. Sanitizers that are large or require an electric outlet are not easily carried around with the electronic devices, and therefore sanitizing is not available to a user when sanitizing is most needed, when the user and the cellphone are away from the home or office. Pre-moistened wipes either dry out prematurely or are packaged in individual sealed envelopes that are cumbersome to carry and difficult to find when needed. Existing portable spray products contain harsh or allergenic ingredients or alcohol, all of which can have undesired consequences for the electronic device or for the user. Portable spray products can also be cumbersome to carry and difficult to find at the time a person wants to use them.

U.S. Pat. No. 6,881,273, issued Apr. 19, 2005 to Charles D. Oberman, covers an “Eyewear Cleaning Device.” The eyewear cleaning device and method provides convenient cleaning of various types of eyewear, such as glasses and sunglasses. The eyewear cleaning device comprises a housing with a retractable cord, at the end of which is attached a soft-nonabrasive cloth for cleaning eyewear lenses. A cleaning fluid dispenser may be included with or attached to the housing. The device can be attached to clothing or other items in proximity to the user of the device, such as a dashboard, sports bag, or backpack.

U.S. Pat. No. 5,083,661, issued Jan. 28, 1992 to Carlton Burwell on a “Lens and/or Eyeglasses Cleaning Device,” covers a device for cleaning eyeglasses wherein a soft, non-abrasive cleaning cloth can be easily stored in, and removed from, an elongated storage container. A retaining cord is attached near the center of the cloth. The cord extends through the main aperture of the storage container, passes through a small hole at the end of the container opposite the aperture, and is terminated with a stopper bead. The cloth is easily retracted into the storage container by pulling on the cord at the stopper bead end. The cloth is of such dimensions that when it is fully retracted into the storage container, a short tail extends through the aperture. The cloth is removed by pulling on the tail. The stopper bead can be a cap for the aperture or act to latch a hinged lid for the aperture. The Burwell patent discloses that when the cloth is in the deployed position, it may be used for cleaning purposes. The container, snap clip, and cap of the apparatus are small and light and do not hinder one from using the cleaning cloth to clean the pair of eyeglasses. The cleaning cloth, container, snap clip, and plastic cap are constrained to the chord in the order listed.

U.S. Pat. No. 2,204,947 issued Jun. 18, 1940 to H. Appelbaum on a “Cosmetic Wiper.” The dominant object of the invention resides in the provision of an improved inexpensive article sustaining means comprising a handkerchief adapted to be utilized as a cosmetic wiper, and suspended from suitable cord means slidable guiding an appropriate jacket adapted to be displaced longitudinally of the cord means and relative to the handkerchief for convenient covering or uncovering. The wiper includes (1) a cord having a stop disposed intermediate the upper and lower portions thereof; (2) a hollow jacket having an upper portion including guide means threadably receiving and slidably guiding said cord; and (3) a handkerchief comprising corner portions and having a central zone portion carried by said lower portion of said cord, adapted to be retractively moved into the jacket upon the application of a thrust to the cord in a direction away from the upper end, where the zone portion cooperates with the guide means to limit the retractive movement of the handkerchief into the jacket to prevent entrance of the corner portions into the jacket during the retractive movement. The jacket has a lower rim cooperating with the handkerchief upon the retractive movement for automatically folding the handkerchief, with the jacket having sides frictionally cooperating with the handkerchief in the retracted relation of the handkerchief to prevent accidental withdrawal of the handkerchief from the jacket. The corner portions in the fully retracted relation of the handkerchief extend beyond the rim and outside of the jacket and constitute manipulatable means for withdrawing the handkerchief outside of the jacket.

U.S. Pat. No. 4,854,449 issued Aug. 8, 1989 to Michael A. Fitzhugh on a “Container in Which to Carry a Resuable Lens Cleaning Material.” The container has a hollow body that provides a dirt-free, moisture resistant environment in which a soft, reusable lens-cleaning (e.g., chamois) material is removably located. A detachable end plug is received within an open end of the container. The end plug is connected to the lens cleaning material by way of a length of chain and an associated connector. Pulling the end plug outwardly from the open end of the container correspondingly causes the cleaning material to be removed therefrom. After use, the material is returned to the interior of the container and the end plug is reattached to the open end thereof. The container is of a suitably compact size so as to be easily carried within a handbag or pocket of the user to provide a convenient and readily available means for cleaning the lens of a pair of glasses, a camera, and the like.

U.S. Pat. No. 8,043,972, issued Oct. 11, 2011 to Janet Cherry, discloses a “Portable, Multi-Purpose Golf Apparatus.” The apparatus includes a casing, a cleaning device, and an opening. The cleaning device may be extendable through the opening an effective distance to clean a golf ball. A carrier may be mounted to the cleaning device within the casing. The carrier may slide within the casing to transition the cleaning device from a stored position to an extended position. The golf apparatus may further include a positioning mechanism coupled to the carrier. The apparatus may include a second opening through which a divot tool or other supplemental device may be extendable.

U.S. Publication No. 2009/0211050, published Aug. 27, 2009 by Priscilla Hawkins for a “Wipe Dispenser,” discloses a handheld device for cleaning surfaces. The improved device comprises a compartment for used wipes and is attachable to the bottom of the dispenser. The storage compartment may have a lid to open the compartment and empty the compartment. The storage compartment may also be fully detachable from the clean tissue unit and fully disposable. Alternatively, the detached compartment may be emptied and reattached to the clean tissue unit. The handheld cleaning device comprises a clean wipe compartment and a storage compartment, where the clean wipe compartment comprises a sliding door that opens and closes a window, allowing exposure of a clean wipe to a desired surface, and the storage compartment is attachable to the clean wipe compartment by twisting or snapping, and further comprises an opening through which a used wiped is inserted into the storage compartment.

U.S. Publication No. 2009/0056048 was published Mar. 5, 2009 by Nima Sharifi for a “Textured Cleaning Wipe for Electronic Devices.” The cleaning wipe has a plurality of stiff triangular or rectangular-cross-sectional projections rising from the surface of a non-woven fabric, designed to clean the nooks and crannies, various crevices, and other hard to reach areas of cell phones or other electronic items with a myriad of buttons and/or camera lenses, charger outputs, mouthpieces, ear receivers, or keypads, which may be designed in countless shapes and sizes. The projections, whether in the form of cones, pyramids, length-long ridges, or other embodiments, are specifically designed to clean the small crevices, the mouthpiece and earpiece, and between small buttons such as keypads on a cell phone or other electronic device.

U.S. Pat. No. 6,194,470, issued Feb. 27, 2001 to Anita Lutz et al., covers a “Sparkle Linen Spray” and discloses a sprayable composition in a pump spray bottle that includes a fragrance oil and reflective material. The reflective material is sized to pass through the atomizer of the pump spray bottle. Linens or clothing can be sprayed with the composition to provide a pleasant smell and sparkling appearance to the surfaces sprayed. It was an object of the Lutz invention to provide a fragrant composition having a novel appearance when dispensed on linens. The fragrance oil and glitter of the apparatus are dispersed in a clear mixture of suspending agent, a non-ionic surfactant to solubilize the fragrance oil, a clarifying agent, propylene glycol, denatured ethyl alcohol, and deionized water with an appropriate pH balancer.

U.S. Publication No. 2002/0190075, published Dec. 19, 2002 by Reinhold Weldi et al., discloses a “Multiple Dispensing Container System” that includes an external housing and a container insert disposed therein. The external housing may be formed from an upper container unit section and a lower container unit section that releasably attach to each other. The container insert is provided with a plurality of cavities adapted for holding and releasing small articles, such as pills or medications.

U.S. Publication No. 2008/0023575, published Jan. 31, 2008 by James Trumble et al. for a “Body Spray Applicator,” discloses a personal body spray applicator having a rectangular shaped body defining a reservoir for containing a liquid. The body has a front face, an opposing back face, and an edge surface adjoining the front face and the back face. A nozzle in fluid communication with the reservoir is provided along the edge surface of the body. An actuator is also positioned along the edge surface of the body and is operable to cause fluid present within the reservoir to be discharged through the nozzle.

U.S. Publication No. 2012/0214878 was published Aug. 23, 2012 by Donald R. Korb for a “High Alcohol Content Stabilizer.” The publication discloses an antimicrobial sanitizing composition in the form of a gel or cream that contains an alcohol in an amount of at least 55% by weight. The sanitizer is capable of disinfecting and providing prolonged antimicrobial property to a variety of surfaces, including the skin, without causing drying, cracking, redness, or irritation. In addition to alcohol, the sanitizer contains an anionic phospholipid in a controlled concentration, as well as components conventionally found in skin creams and gels such as emulsifiers, emollients, moisturizers, rheology modifiers, and neutralizers. The anionic phospholipid enables use of the composition without the user experiencing dryness or irritation and the feeling of a greasy residue on the skin following use.

There thus is a need for an electronic-device sanitizer product and method that is easy to carry, easy to find when needed, and easy to use. Such a sanitizer would travel with the cellphone or electronic device, which would allow frequent cleaning and would allow cleaning when the user is out in public and most needs to sanitize the electronic device. Such a sanitizer would not contain alcohol or any harsh or allergenic ingredients. And such a sanitizer would not smell offensive to the user, but would smell either of nothing or of a desired scent chosen by the user.

### SUMMARY OF THE INVENTION

This invention provides an electronic-device sanitizer product and method for sanitizing cellphones, smartphones, and other portable electronic devices, that is easy to carry, easy to find when needed, and easy to use, allowing for frequent cleaning, having an alcohol-free sanitizing liquid, a misting sprayer, and a microfiber material for distributing and absorbing the sanitizing liquid, all enclosed in a slim tube-shaped package that can be carried on a keyring. Optionally, the sanitizing liquid can be provided with any of a number of distinctive scents that will be imparted to the electronic device.

### BRIEF DESCRIPTION OF DRAWINGS

Reference will now be made to the drawings, wherein like parts are designated by like numerals, and wherein:

FIG. 1 is an axonometric view of the electronic-device sanitizer;

FIG. 2 is an axonometric view of an embodiment of the electronic-device sanitizer having a hollow compartment in the unit body for storage of the microfiber material;

FIG. 3 is an axonometric view of an embodiment of the electronic-device sanitizer having the microfiber material attached to and rolled around the unit body when not in use;

FIG. 4 is an axonometric view of an embodiment of the electronic-device sanitizer having the microfiber material attached to and forming the outside surface of the unit body and unit cap at all times;

FIG. 5 is an axonometric view of an embodiment of the electronic-device sanitizer having the microfiber material attached to and forming the surface of the bottom of the unit body at all times; and

FIG. 6 is an axonometric view of an embodiment of the electronic-device sanitizer having a cushion of the microfiber material extendibly and retractably mounted in the bottom of the unit body;

### DETAILED DESCRIPTION OF THE INVENTION

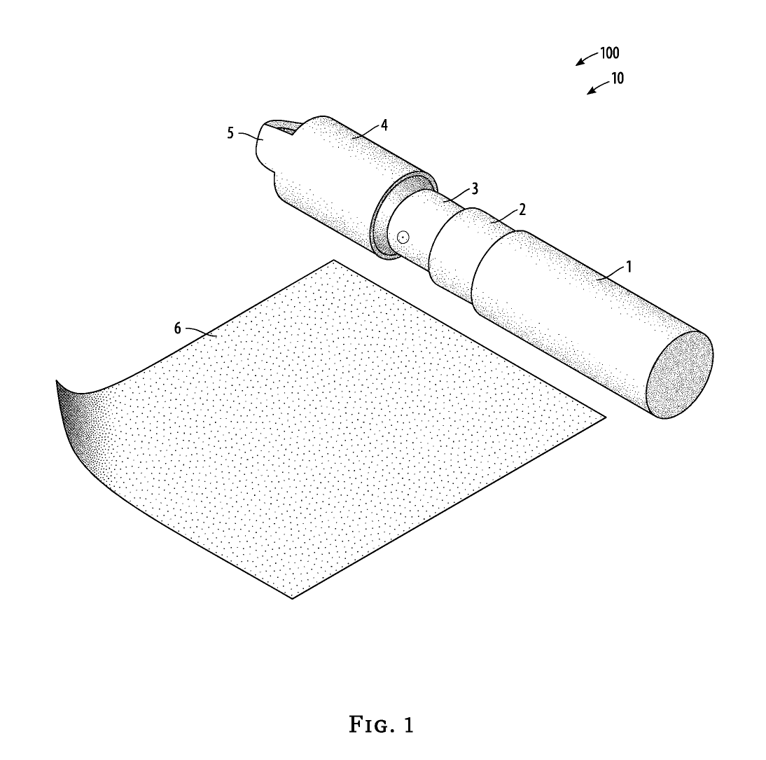

Referring to all figures generally, embodiments of the invention method 100 and product 10 are illustrated. Referring to FIG. 1, the elements of the electronic-device sanitizer are a unit body 1, a liquid reservoir 2, a liquid sprayer 3, a unit cap 4 having a keychain attachment structure 5, and a piece of microfiber material 6.

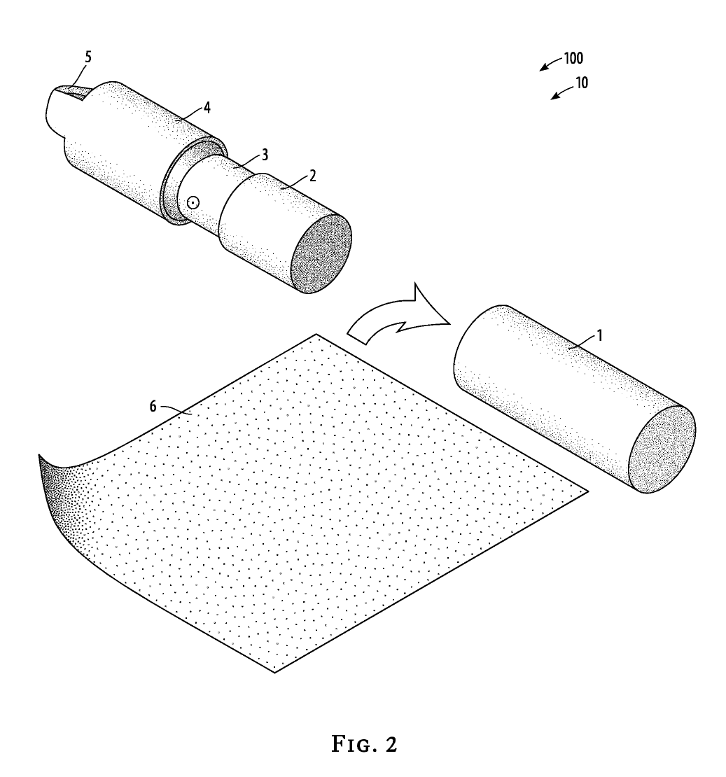

Referring to FIG. 2, in an embodiment of the electronic-device sanitizer, the unit body 1 has an accessible hollow compartment that the microfiber material 6 can be stored in when the invention is not in use.

Referring to FIG. 3, in another embodiment of the electronic-device sanitizer, the microfiber material 6 is wound around the unit body 1 for storage and is unwound for use.

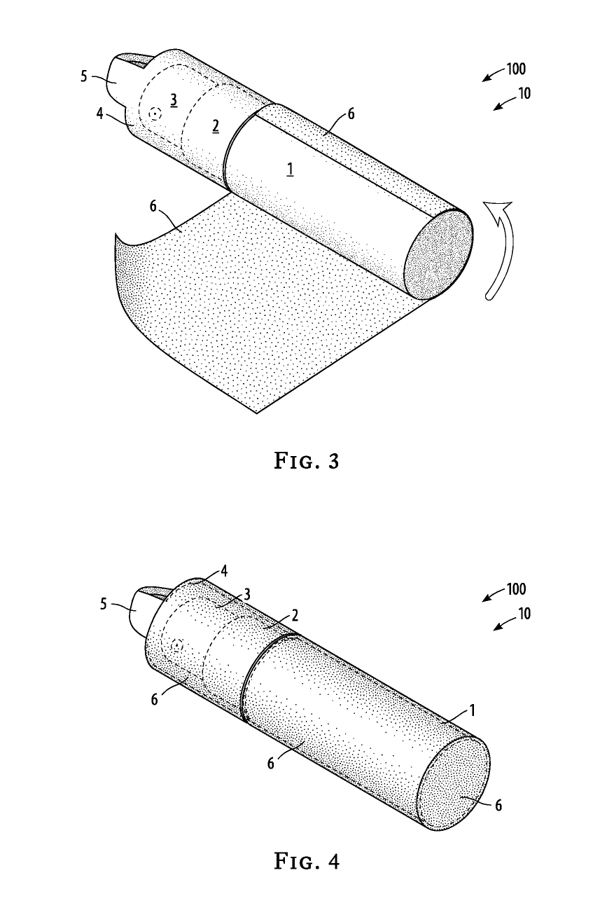

Referring to FIG. 4, in another embodiment of the electronic-device sanitizer, the microfiber material 6 is attached to the outside surface of the unit body 1 and the outside surface of the unit cap 2, forming an outer surface of microfiber material 6 at all times.

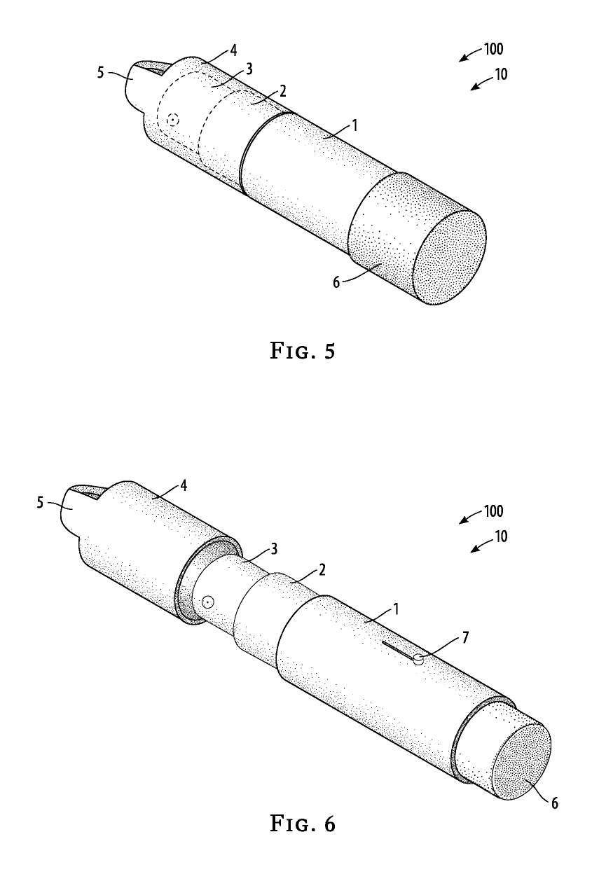

Referring to FIG. 5, in another embodiment of the electronic-device sanitizer, the microfiber material 6 is attached to and forms the surface of the bottom of the unit body 1 at all times.

Referring to FIG. 6, in another embodiment of the electronic-device sanitizer, the microfiber material 6 is provided as a cushion that is contained in the bottom of the unit body 1 and can be extended for use and retracted for storage by the operation of a sliding device 7.

The form of the embodiments of the electronic-device sanitizer make the invention easy to carry because of the small size, the storage of all elements either in or on the single unit, and the provision of a keychain attachment structure 5. The invention is easy to find when needed because of the storage of all elements either in or on the single unit, and the provision of a keychain attachment structure 5. The invention is easy to use because the liquid sprayer 3 and the microfiber material 6 are easily accessed, and because the sanitizing liquid is formulated to be safe for the user and for the electronic device, non-irritating, effective in small quantity as a mist, and easily distributed on the surface of the device and removed from the surface of the device with the microfiber material 6. It is not critical that all of the sanitizing liquid be removed from the surface of the device, but the removal of fingerprints and other smudges requires some wiping with the microfiber material 6.

The sanitizing liquid can be sprayed as a mist directly onto the electronic device or can be sprayed onto the microfiber material 6 and transferred from the microfiber material to the surface of the electronic device, as best suits specific devices and specific users.

The sanitizing liquid comprises a carrier liquid and a sanitizing mixture. In a preferred embodiment, the sanitizing mixture comprises 100 parts Essential Oil having the desired scent and antimicrobial qualities, 8 parts Castille soap, 4 parts Sunflower Oil, 4 parts Glycerine, and 1 part Xanthan Gum. These ingredients do not have any allergenic effect on most people, and are used for their antimicrobial, cleaning, and emulsifying properties.

The carrier liquid comprises corn starch dissolved in water in the ratio of approximately 0.1 milliliters of corn starch to 1 liter of water. The corn starch reduces streaking of the cleaned surfaces. To this carrier liquid is added the sanitizing mixture in the ratio of 0.3 milliliters of sanitizing mixture to 1 liter of carrier liquid.

Optionally, sanitizing liquid can be provided in a variety of scents according to the essential oil used. Such a variety of scents allows users to choose a distinctive scent and to vary the scent according to season, holidays, or other motivation.

Many changes and modifications can be made in the present invention without departing from the spirit thereof. I therefore pray that rights to the present invention be limited only by the scope of the appended claims.

What is claimed is:

1. An electronic-device sanitizer product, comprising:
(i) a unit body of essentially cylindrical form having a long dimension of between 1 and 3.5 inches, inclusive, and a diameter of between 0.25 and 1.3 inches, inclusive, having a nominal top, bottom, and side;
(ii) a unit cap of essentially cylindrical form having a diameter the same as or nearly the same as said unit body, having a long dimension of at least 0.5 inches, and sufficient to make a total combined unit body plus unit cap length of between 2 and 6 inches, inclusive, having a nominal top, bottom, and side, adapted to be removed from and reconnected to said unit body at the bottom of said unit cap at the top of said unit body;
(iii) a keychain attachment structure affixed to the top of said unit cap, adapted to attach to a keychain, carabiner, or other hook;
(iv) a liquid reservoir adapted to fit on to or into said unit body at the top of said unit body, and adapted to fit within said unit cap;
(v) a liquid sprayer adapted to spray liquid from said liquid reservoir in a fine mist; and
(vi) a microfiber material adapted to absorb and distribute liquid sprayed from said liquid sprayer, and adapted to be carried on or inside of said electronic-device sanitizer;
where said electronic-device sanitizer is easily carried by and accessible to a person carrying an electronic device;
where said liquid reservoir is filled with an alcohol-free sanitizing liquid, the sanitizing liquid further comprising:
(i) a carrier liquid comprising 10,000 parts water and 1 part corn starch; and
(ii) a sanitizing mixture added to said carrier liquid in the ratio of 3 parts sanitizing mixture to 10,000 parts carrier liquid;
where said sanitizing liquid is adapted to be sprayed in a mist upon the exterior surfaces of an electronic device or upon said microfiber material for transfer to the electronic device, to clean and sanitize the electronic device without damaging the electronic device and without irritating the person carrying the electronic device; and
where said microfiber material is used to distribute and absorb said sanitizing liquid from the exterior surfaces of the electronic device without leaving any residue or mark.

2. The electronic-device sanitizer product of claim 1, where said sanitizing mixture further comprises 100 parts Essential Oil having the desired scent and antimicrobial qualities, 8 parts Castille soap, 4 parts Sunflower Oil, 4 parts Glycerine, and 1 part Xanthan Gum.

3. The electronic-device sanitizer product of claim 1, where said sanitizing liquid further provides a distinctive scent to the sanitized electronic device.

4. The electronic-device sanitizer product of claim 1, where said liquid sprayer is a spray pump.

5. The electronic-device sanitizer product of claim 1, where said microfiber material further comprises a sheet of material adapted to be placed into said unit body when not in use, and where said unit body further comprises a hollow compartment adapted to receive said microfiber material when not in use.

6. The electronic-device sanitizer product of claim 1, where said microfiber material further comprises a sheet of material adapted to be placed around the exterior of said electronic-device sanitizer when not in use.

7. The electronic-device sanitizer product of claim 1, where said microfiber material further comprises material affixed to the exterior of said unit body.

8. The electronic-device sanitizer product of claim 1, where said microfiber material further comprises material permanently affixed to the exterior of the bottom of said unit body.

9. The electronic-device sanitizer product of claim 1, where said microfiber material further comprises a retractable cushion, and where said unit body further comprises a sliding device adapted to extend and retract said microfiber material.

10. The electronic-device sanitizer product of claim 1, where the combined length of said unit cap attached to said unit body is in the range from 3 inches to 4 inches, inclusive.

11. An electronic-device sanitizer method, comprising:
(i) providing an electronic-device sanitizer product, comprising:
(a) a unit body of essentially cylindrical form having a long dimension of between 1 and 3.5 inches, inclusive, and a diameter of between 0.25 and 1.3 inches, inclusive, having a nominal top, bottom, and side;
(b) a unit cap of essentially cylindrical form having a diameter the same as or nearly the same as said unit body, having a long dimension of at least 0.5 inches, and sufficient to make a total combined unit body plus unit cap length of between 2.5 and 6 inches, inclusive, having a nominal top, bottom, and side, adapted to be removed from and reconnected to said unit body at the bottom of said unit cap at the top of said unit body;
(c) a keychain attachment structure affixed to the top of said unit cap, adapted to attach to a keychain, carabiner, or other hook;
(d) a liquid reservoir adapted to fit on to or into said unit body at the top of said unit body, and adapted to fit within said unit cap;
(e) a liquid sprayer adapted to spray liquid from said liquid reservoir in a fine mist; and
(f) a microfiber material adapted to spread and absorb liquid sprayed from said liquid sprayer, and adapted to be carried on or inside of said electronic-device sanitizer;
where said electronic-device sanitizer is easily carried by and accessible to a person carrying an electronic device;
(ii) filling said liquid reservoir is with an alcohol-free sanitizing liquid, the sanitizing liquid further comprising:
(a) a carrier liquid comprising 10,000 parts water and 1 part corn starch; and
(b) a sanitizing mixture added to said carrier liquid in the ratio of 3 parts sanitizing mixture to 10,000 parts carrier liquid;
(iii) spraying said sanitizing liquid in a mist upon the exterior surfaces of an electronic device or upon said microfiber material for transfer to the electronic device, to clean and sanitize the electronic device without damaging the electronic device and without irritating the person carrying the electronic device; and
(iv) wiping said microfiber material to distribute and absorb said sanitizing liquid from the exterior surfaces of the electronic device without leaving any residue or mark.

12. The electronic-device sanitizer method of claim 11, where said sanitizing mixture further comprises 100 parts Essential Oil having the desired scent and antimicrobial qualities, 8 parts Castille soap, 4 parts Sunflower Oil, 4 parts Glycerine, and 1 part Xanthan Gum.

13. The electronic-device sanitizer method of claim 11, where said sanitizing liquid further provides a distinctive scent to the sanitized electronic device.

14. The electronic-device sanitizer method of claim 11, where said liquid sprayer is a spray pump.

15. The electronic-device sanitizer method of claim 11, where said microfiber material further comprises a sheet of material adapted to be placed into said unit body when not in use, and where said unit body further comprises a hollow compartment adapted to receive said microfiber material when not in use.

16. The electronic-device sanitizer method of claim 11, where said microfiber material further comprises a sheet of material adapted to be placed around the exterior of said electronic-device sanitizer when not in use.

17. The electronic-device sanitizer method of claim 11, where said microfiber material further comprises material affixed to the exterior of said unit body.

18. The electronic-device sanitizer method of claim 1, where said microfiber material further comprises material permanently affixed to the exterior of the bottom of said unit body.

19. The electronic-device sanitizer method of claim 11, where said microfiber material further comprises a retractable cushion, and where said unit body further comprises a sliding device adapted to extend and retract said microfiber material.

20. The electronic-device sanitizer method of claim 11, where the combined length of said unit cap attached to said unit body is in the range from 3 inches to 4 inches, inclusive.
